(19) United States
(12) Patent Application Publication
(10) Pub. No.: US 2022/0409766 A1
(43) Pub. Date: Dec. 29, 2022

(54) SANITIZING FACE MASK

(71) Applicants: Donald SPECTOR, Jupiter, FL (US); Lisa PAMINTUAN, Jupiter, FL (US)

(72) Inventors: Donald SPECTOR, Jupiter, FL (US); Lisa PAMINTUAN, Jupiter, FL (US)

(21) Appl. No.: 17/903,153

(22) Filed: Sep. 6, 2022

Related U.S. Application Data
(63) Continuation-in-part of application No. 17/509,279, filed on Oct. 25, 2021.
Application No. 17/509,279 is a continuation-in-part of application No. 17/097,132, filed on Nov. 13, 2020.
(60) Provisional application No. 63/196,775, filed on Jun. 4, 2021.
(60) Provisional application No. 63/105,248, filed on Oct. 24, 2020.
(60) Provisional application No. 63/298,962, filed on Jan. 12, 2022.

(51) Int. Cl. A61L 9/20; A41D 13/11

(52) U.S. Cl. CPC A61L 9/20; A41D 13/11; A61L 2209/12

(57) ABSTRACT

A face mask is connected via a tube to a housing containing an ultraviolet (UV) light-emitting diode (LED) in a channel. A power source is connected to the UV LED. The channel is helical in shape and has undulations, so as to maximize the amount time the air is subjected to the UV radiation. The housing rear face has a fins to allow the heat from the LEDs to dissipate through the fins during use. This arrangement protects the user and others from excessive UV exposure and heat during use.

### CROSS-REFERENCE TO RELATED APPLICATIONS

This application is a continuation-in-part of U.S. patent application Ser. No. 17/509,279, filed on Oct. 25, 2021, which claims priority under 35 USC 119e of U.S. Provisional Application No. 63/196,775 and which is a continuation-in-part of U.S. patent application Ser. No. 17/097,132, filed on Nov. 13, 2020, which claims priority under 35 USC 119(e) of U.S. Provisional Patent Application No. 63/105,248, filed on Oct. 24, 2020, the disclosures of all of which are herein incorporated by reference. This application also claims priority under 35 USC 119(e) of U.S. Provisional Application Ser. No. 63/298,962, filed on Jan. 12, 2022, the disclosure of which is herein incorporated by reference.

### BACKGROUND OF THE INVENTION

### 1. Field of the Invention

The present invention relates to a sanitizing face mask which utilizes ultraviolet light to sterilize the wearer's exhalations as they are emitted from the mask.

### 2. The Prior Art

The respiratory system represents a closed air system with a self-contained cavity, much smaller than a car cabin, or room. Unlike man-made rooms or vehicles in which air ducts and fans are used to circulate air, the human body uses inhalation and expiration as a mechanism to fill the respiratory system, thereby providing needed oxygen to the blood supply.

The respiratory system is also a pathway into the body for viruses, bacteria and other harmful pathogens. Inhalation can bring such organisms into the person and exhalation can deliver particles containing such organisms to others, thereby spreading disease.

Face masks are often worn by medical professionals to protect patents from any viruses or bacteria that may be harbored by the wearer. The face masks also provide a degree of protection to the wearer from outside sources. These masks can be made of a variety of materials, such as paper, fabric or various nonwoven materials. While these masks can be somewhat effective in preventing the virus particles from escaping through the mask, their efficacy is limited due to an imprecise fit and limited filtration. it would be desirable to equip the masks with a way to kill the virus particles so that even those that escape from the mask cannot infect the public.

### SUMMARY OF THE INVENTION

This object is accomplished according to the invention by a sanitizing device comprising a housing having a central aperture and an ultraviolet (UV) light-emitting diode (LED) assembly mounted in the housing. The UV LED assembly is formed of a plurality of connected UV LEDs arranged circumferentially around the central aperture so as to project UV-C light into the aperture. UV-C light is a short-wave ultraviolet light that has been found to be effective in killing viruses in the air and on surfaces. Far UV-C light (e.g. 222 nm wavelength) is preferably used, as exposure to it has less adverse effects on humans. Alternative to an LED assembly, other sources of UV-C light could also be used. A power source is connected to the UV LED assembly to supply power to the assembly. The housing is configured with overhanging front and rear faces, so that the UV LED assembly is recessed within the housing and is not visible when viewing the housing directly from the front or rear. This arrangement protects the user and others from excessive UV exposure during use.

There is an attachment layer connected to a rear surface of the housing. The attachment layer is configured for attaching the sanitizing device to a face mask. The attachment layer can be any suitable attachment means, such as a releasable adhesive or a hook-and-loop type fastener, that follows the toroidal shape of the housing. If a hook-and-loop type fastener is used, one side of the fastener is affixed to the housing, and then a plurality of corresponding layers of the other side of the fastener are provided for attachment to a face mask, as new fastener is required each time the mask is discarded an a new one is used. Each side of the fastener can be affixed to the housing or the mask via an adhesive substance. The power source also has an attachment layer connected to its back surface. The attachment layer can also be any suitable attachment means, such as an adhesive or hook-and-loop type closure. A switch can be connected to the housing to turn the LEDs on and off. An indicator light can be connected to the switch so that the user, who may not be able to see the LEDs that are recessed in the housing, will know whether the LEDs are on or off.

To further prevent exposure by the LEDs, a filter cover can be placed over the aperture in the housing, on the front and/or rear sides of the housing.

The power source can be any suitable source of electrical power for the LEDs. In one embodiment, the power source is a battery. The battery is preferably configured to be as small and as flat as possible, so that it can be easily adhered to a face mask. The battery is connected to the LED assembly via a wire.

The sanitizing device according to the invention is ideally used on disposable paper face masks to sanitize the user's inhalations and exhalations. The housing is affixed to a front or rear central portion of the face mask, adjacent the wearer's mouth, so as to capture the majority of air exhaled by the wearer. The battery can be affixed to any suitable location on the mask. Once the mask has been used, the sanitizing device can be removed from the mask and re-used on a new mask by affixing the housing and battery to the new mask. The sanitizing device can be re-used as many times as needed. The battery can be replaced when it is depleted. Alternatively, a re-chargeable battery can be used instead. While envisioning a lightweight battery that can be connected to the housing and also adhered onto the mask, the invention does not require a particular design with regard to the battery. Therefore, for more professional uses it may be required to have more LEDs and a greater battery supply, in which case the wire can be connected to a larger battery source that can be located or clipped onto, held or stored on the person wearing the mask as well as any person or object in proximity to the mask, such that it is electrically connected to the sanitizing device according to the invention.

In an alternative embodiment, the sanitizing device can be permanently mounted in an aperture in a face mask so that it is not removable, or can be retro-fitted into a valve nozzle in an existing mask. The power source can be in the form of a battery that is attached to the mask, or can be in the form of a conductive material such as graphene, that is printed on the mask itself and connected to the LED assembly through the material of the mask.

In another embodiment of the invention, the mask is in the form of a rigid outer shall that is equipped with a closed tube through which the user breathes. The tube opens at an inlet opening through which air enters the tube from outside the outer shall, and extends in a serpentine pattern though the mask to an outlet opening adjacent the user's mouth and nose. The UV-C light sources, which can be LEDs, are positioned within the tube, so that air going in and coming out of the tube is exposed to the UV light in both directions. The user's skin is completely insulated from the UV light, as the LEDs are only located inside the closed tube. An additional face shield can be placed over the tube to further insulate the user's face from the radiation. The face shield can have an opening to accommodate the outlet opening of the tube.

While this invention calls for inhalation and exhalation to be the driving force in moving air in and out of the body, the invention is not limited thereby.

The light can be amplified by parabolic reflectors or mirrors and sealed from light leakage to the skin or eyes. Excessive heat caused by the LEDs is reduced due to the air flow through the tube during inhalation and exhalation. The lungs act as an air pump, thereby replacing the need for a fan by the respiratory system itself. Hence, when proper heat dissipating materials are used, the actual heat only creates a slight elevation in the temperature of the air entering the lungs, which is also desirable for the respiratory system. Therefore, the respiratory system itself absorbs the heat energy of the diodes using the face mask as an extension of the respiratory system while also accomplishing no light contact with the skin or eyes at the same time.

Multiple LEDs can be placed along the extent of the tube. Heat sinks can be attached to the LEDs to further reduce the amount of heat from the LEDs that is released into the region of the mask. The serpentine shape of the tube with multiple curves ensures that air that is inhaled and exhaled is exposed to radiation for a longer period of time than with the use of a shorter, straight tube. The tube can be removable from the outer shell or can be integrally molded with the outer shell.

In a further embodiment, the LEDs are contained in a separate housing that is connected to the mask by tubing. The mask can be formed of any suitable material, such as paper, flexible plastic, or a hard plastic shell. A ventilation tube is connected to the mask and extends to a separate housing that has internal channels inside. Air exhaled from the user travels through the tubing and into the housing, where it travels through the internal channels and out of an exit port in the housing. LEDs are arranged in the internal channels, and are powered by a power source such as batteries, which are also contained in the housing. As the air travels through the channels, it is irradiated by the LEDs, so that viruses, bacteria, etc. are destroyed prior to the air exiting from the exit port. In addition, air that is inhaled by the user enters the housing through the exit port, travels through the channels and the tube to the user. As it does so, the air entering the housing from the outside is also irradiated by the LEDs so that the user is inhaling purified air as well.

In addition to the UV radiation, the LEDs also emit heat, which can also aid in destroying the viruses or bacteria. However, an additional heat source could also be supplied to the channels, in addition to the LEDs. While a single tube can be used for both inhalation and exhalation, two separate tubes could also be used, and connected to the housing by different ports. The two tubes could merge into a single tube prior to connection to the mask, and be closed off from each other by a valve that is activated by the user's breathing patterns. The housing could be attached to a strap or other connecting devices to make it easy for the user to carry. The housing could also be attached to the user via straps or other devices.

In a further embodiment, the housing is constructed of several parts that fit together to create the channels. The housing is formed by a back panel to which the LEDs are affixed, a frame structure surrounding the back panel, a channel plate having at least one channel arranged therein, and a front panel with an exit/entry port. The frame structure connects to the back panel and to the front panel, keeping the channel plate between them. The LEDs are arranged on the back panel so as to be disposed in the at least one channel of the channel plate when the housing is assembled.

In a preferred embodiment, the channel is arranged in a helical structure, so that the inhaled/exhaled air travels in a helical path, from outside to inside. At the center of the channel plate is a relief opening that allows air to enter and be expelled during use.

The channel, in addition to having a helical structure, can have meandering side walls that elongate the channel as well as create eddies as the air flows through it. In particular, the side walls can be constructed so as to create rounded bulges throughout the helical course of the channel, to trap the air and cause the eddies. This way, the air is maintained exposed to the LEDs for a longer period of time, allowing further disinfection of the air entering and exiting the housing.

The back panel can have an exterior surface that has a series of fins, forming a radiator structure, thus increasing the surface area of the back plate, to allow heat from the LEDs to dissipate more quickly, and not overheat the interior of the housing. The fins can be separated by spacers that allow the housing to be placed on a surface without damaging the fins.

It is believed that this invention will add another layer of protection to masks used for mitigation of disease. The invention is comfortable, easy to operate and convenient to use.

### BRIEF DESCRIPTION OF THE DRAWINGS

Other objects and features of the present invention will become apparent from the following detailed description considered in connection with the accompanying drawings. It is to be understood, however, that the drawings are designed as an illustration only and not as a definition of the limits of the invention.

In the drawings, wherein similar reference characters denote similar elements throughout the several views:

FIG. 1 shows an exploded view of the front of the sanitizing device according to the invention;

FIG. 2 shows an exploded view of a rear of the sanitizing device according to the invention;

FIG. 3 show one embodiment of the sanitizing device in use on a disposable face mask;

FIG. 4 shows the embodiment of FIG. 3 being worn by a person;

FIG. 5 shows another embodiment of the sanitizing device mounted in another face mask and worn by a person;

FIG. 6 shows a block diagram of the components of the sanitizing device according to the invention;

FIG. 7 shows an alternative embodiment of the invention in the form of a mask having a sanitizing device built in, and as worn by a user;

FIG. 8 shows an exterior view of the mask of FIG. 7;

FIG. 9 shows an interior view of the mask of FIG. 7;

FIG. 10 shows the view of FIG. 9 with the face shield removed;

FIG. 11 shows a cross-sectional view of the mask, showing the interior of the LED tube;

FIG. 12 shows an alternative embodiment of the mask in cross-section;

FIG. 13 shows another alternative embodiment of the mask;

FIG. 14 shows an enlarged view of the mask element of FIG. 13;

FIG. 15 shows the interior of the housing containing the LEDs in the embodiment of FIG. 13;

FIG. 16 shows an alternative embodiment of the housing in a front view;

FIG. 17 shows the embodiment of FIG. 16 in a rear view;

FIG. 18 shows the embodiment of FIG. 16 in an exploded view;

FIG. 19 shows an opposite exploded view of the embodiment of FIG. 18; and

FIG. 20 show a plan view of the channel plate.

### DETAILED DESCRIPTION OF THE PREFERRED EMBODIMENTS

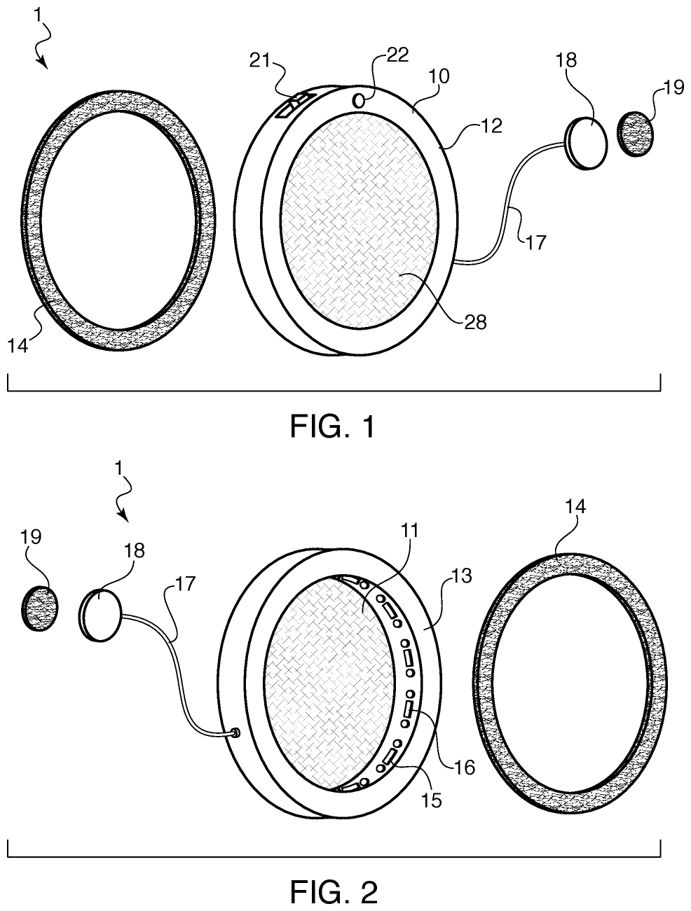

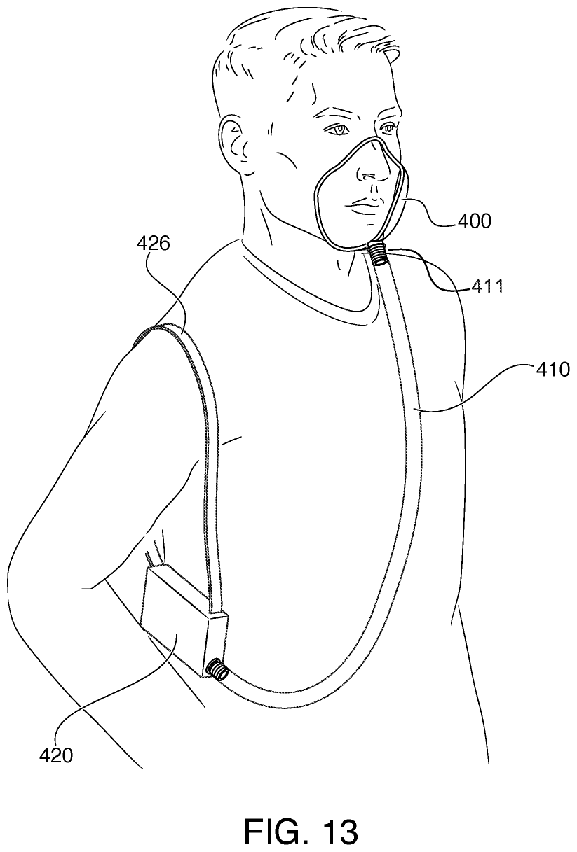

Referring now in detail to the drawings. FIGS. 1 and 2 show an exploded view of the components of the sanitizing device according to the invention. Sanitizing device 1 is formed from a housing 10 having a central aperture 11, overhanging flanges 12, 13 and an attachment layer 14 that is configured to be adhered to the rear flange 13 and attach the housing 11 to a device such as a face mask 20, as shown in FIG. 13. Housing 11 can be made of any suitable lightweight material, such as plastic or aluminum. Inside housing 11 is a UV LED assembly 15, comprised of individual UV LED's 16, connected by a wire 17′ (shown in FIG. 6). UV assembly 15 is recessed behind flanges 12,13 so that the light emitted therefrom is not directed to the user or to other people facing the user. The number of UV LEDs in the assembly can vary based on the user's preferences, the size of housing and the amount of power that is able to be supplied to the device. A cover layer 28, which can be made of any suitable air-permeable material, is placed over central aperture 11. Also connected to UV LED assembly 15 via wire 17 is a power source 18, which can be in the form of a battery. Power source 18 can also have an attachment layer 19, for attachment to face mask 20. Attachment layers 14, 19, can be formed of adhesive, or preferably a hook-and-loop type closure such as what is known as VELCRO®, which has one part affixed to the housing 10 and the other part affixed to the mask 20, so that the two parts can interlock to attach the housing 10 to the mask 20.

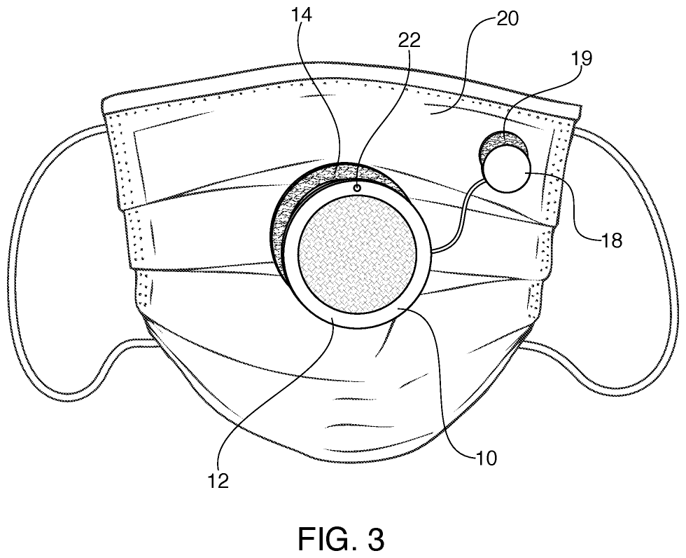

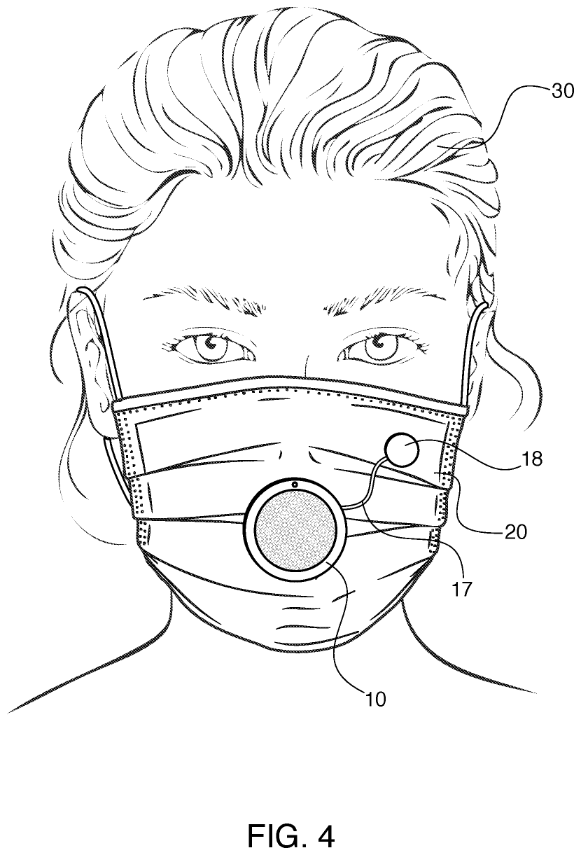

As shown in FIG. 3, housing 10 and power source 18 are attached to the front surface of mask 20, but could also be attached to the rear surface of mask 20. Housing 10 is preferably positioned in the center of mask 20, in front of where a user's 30 mouth would be located, such as shown in FIG. 4. Mask 20 is preferably a disposable surgical mask. Housing 10 and power source 18 can be re-used with different masks, by simply removing attachment layer 14, 19 from the mask when the mask is ready to be discarded, and then applying attachment layers 14, 19 to a new mask. If attachment layers 14, 19 are hook-and-loop type closures, a set of extra closure parts that can be affixed to the mask can be provided, so that a new closure part is applied each time to the new mask.

As shown in FIGS. 1-4, power source 18 is a small flat battery that can be easily applied to face mask 20. However, for other applications, a larger battery can be used, that is not applied to face mask 20 and is instead clipped to the user's belt or other article of clothing. The battery can be disposable or rechargeable.

As shown in the diagram in FIG. 6, switch 21 can be connected to power source 18, to allow the user to turn power on and off to LED assembly 15. In addition, an indicator light 22 can be connected to LED assembly 15, and can be illuminated when power is supplied to LED assembly 15. This is necessary because LED assembly 15 is recessed behind flanges 12, 13 so that light from the LEDs is not easily seen by observers. However, the light from the LED's is projected into central aperture 11 so that any inhalations and exhalation of the user 30 pass through central aperture 11 and are irradiated by UV assembly 15 when the power is on. This helps to decrease the amount of viruses and bacteria that can be transmitted to and from the user via breathing. As shown in the diagram in FIG. 6, a fan can be optionally connected to power source 18, to help move air through central aperture 11.

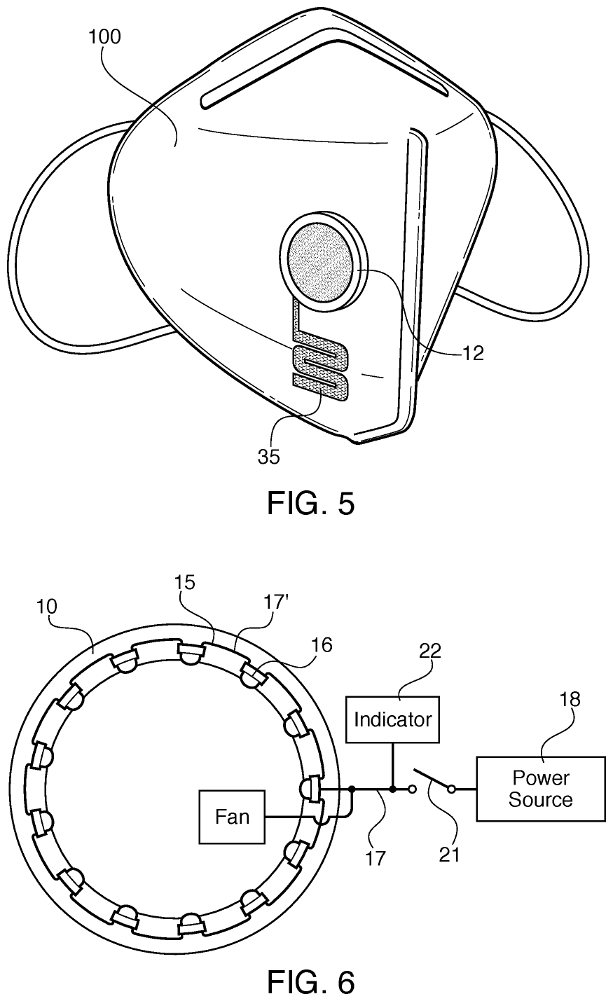

An alternative embodiment of the invention is shown in FIG. 5. Here, housing 10 is mounted in the valve port of a N95 respirator mask 100 make a permanent breathing apparatus. Instead of a removable battery, power source 35 is printed directly on mask 100. Power source 35 can be made of graphene or any other suitable material for supplying power to LED assembly 15. Alternatively, power source 35 could be an auxiliary battery that is clipped to the user's clothing or belt and connected by a wire. This way, a larger battery could be accommodated in the device according to the invention and would allow for the use of a greater number or LED's or a longer battery life.

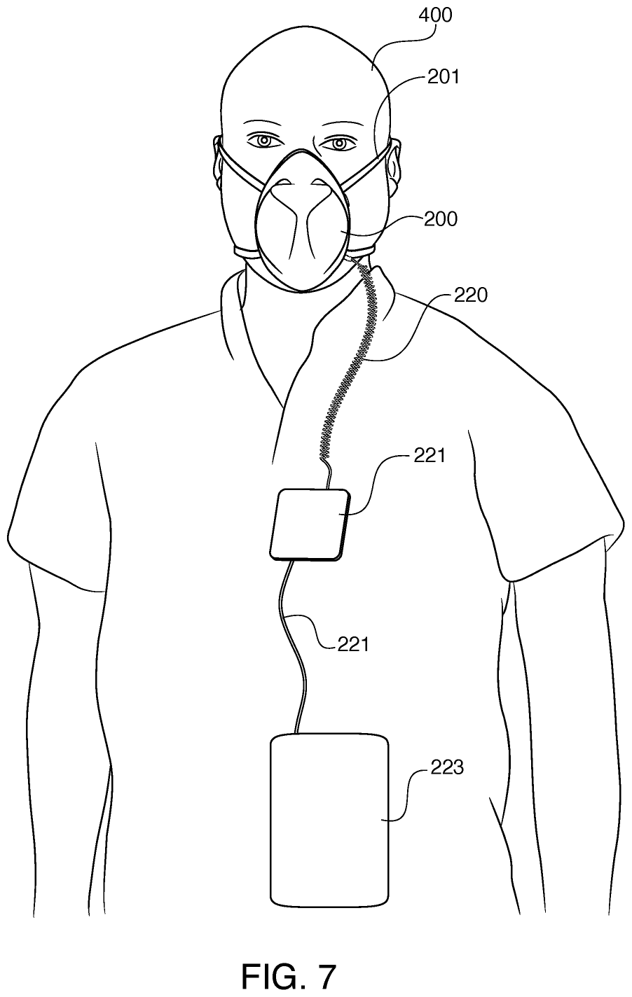

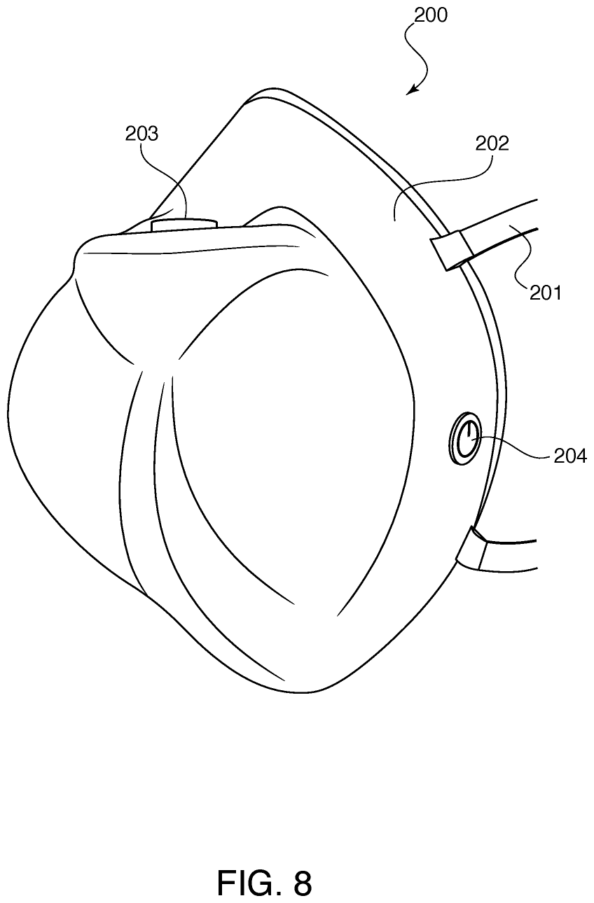

FIGS. 7-11 show alternative embodiments of the invention. Here, face mask 200 is positioned on the face of a user 400 via straps 201. However, other attachment devices could also be used. A cord 220 extends to a controller 221 connected to a battery 223 via a wire 222. However, other power sources and controllers could also be used, including ones that are entirely built into the mask 200. Battery 223 can be used if long duration of mask use is required, as it can have a larger capacity than a battery built into the mask. Alternatively, a mask with both a built-in and auxiliary battery can be used. As shown in FIG. 8, face mask 200 has an outer shell 202 connected to straps 202. An inlet opening 203 is disposed in outer shell 202 to let in air. Inlet opening 203 faces upward. Outer shell 202 is shaped to fit around the nose and mouth of user 400 in a comfortable manner yet fit snugly against the user's face at the edges of mask 200 to prevent air from entering the interior from the edges. Outer shell 202 can be formed of any suitable rigid or semi-rigid material, such as polyethylene. A power button 204 is disposed on mask 200 and connected to controller 221 and battery 223 to turn the LEDs (discussed below) inside on and off.

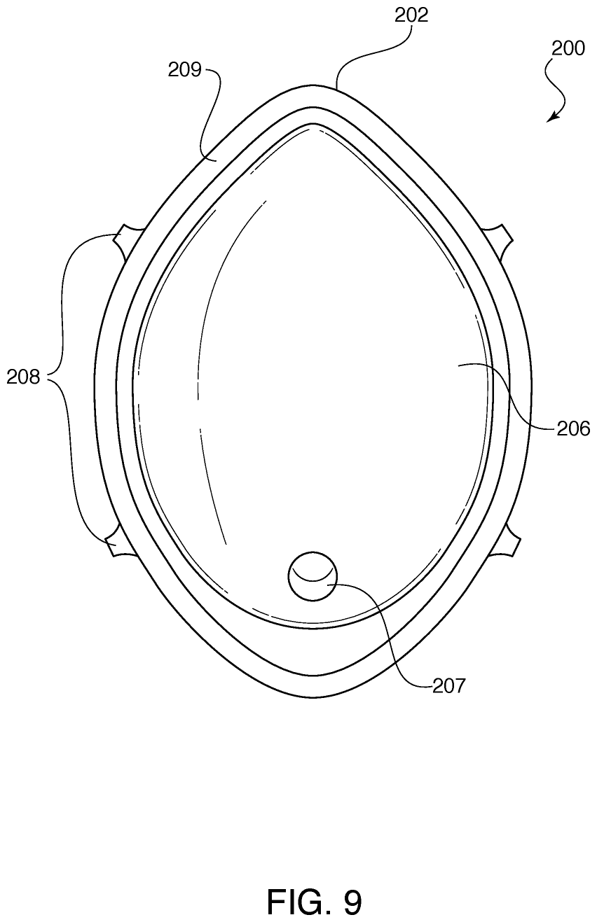

FIG. 9 shows the rear view of mask 200, which can be equipped with a face shield 206 that faces the user's face. Face shield 206 adds further protection against heat and radiation from the LEDs inside. Face shield 206 can be formed of an opaque rigid or pliable material, such as plastic, or woven or nonwoven fabric. An opening 207 provides air flow to the user when the mask is worn. A sealing material such as silicone or rubber can be applied along the edge 209 of mask 200 to enhance the fit and prevent air leakage. Connection points 208 allow for the connection of straps 201 to mask 200 for securing mask 200 to the user's face.

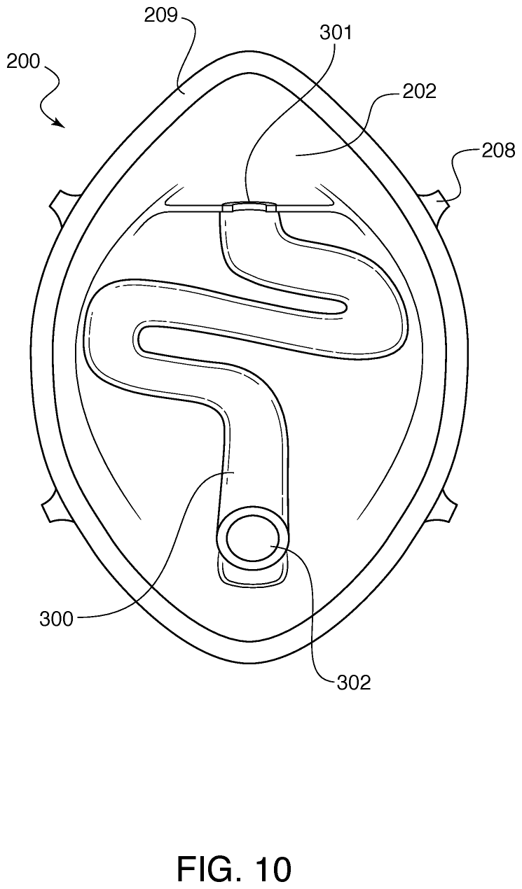

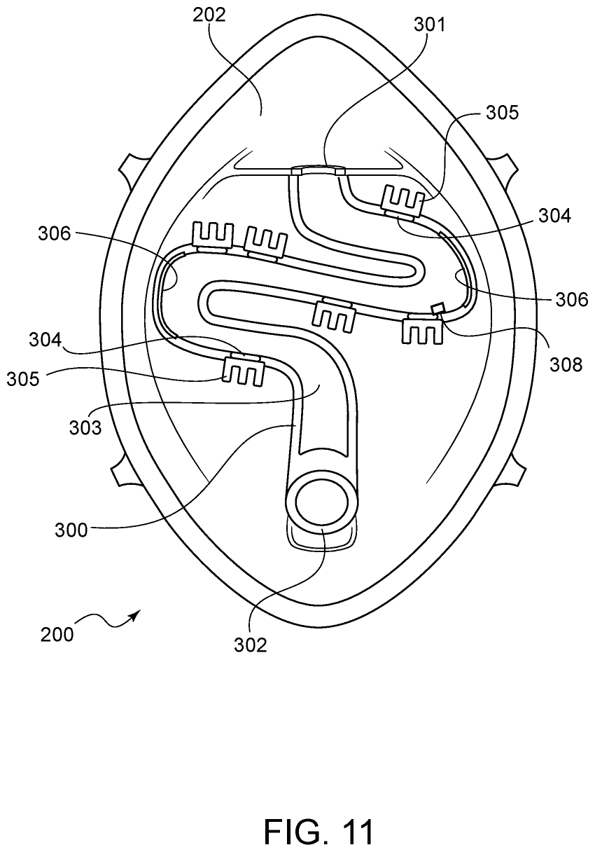

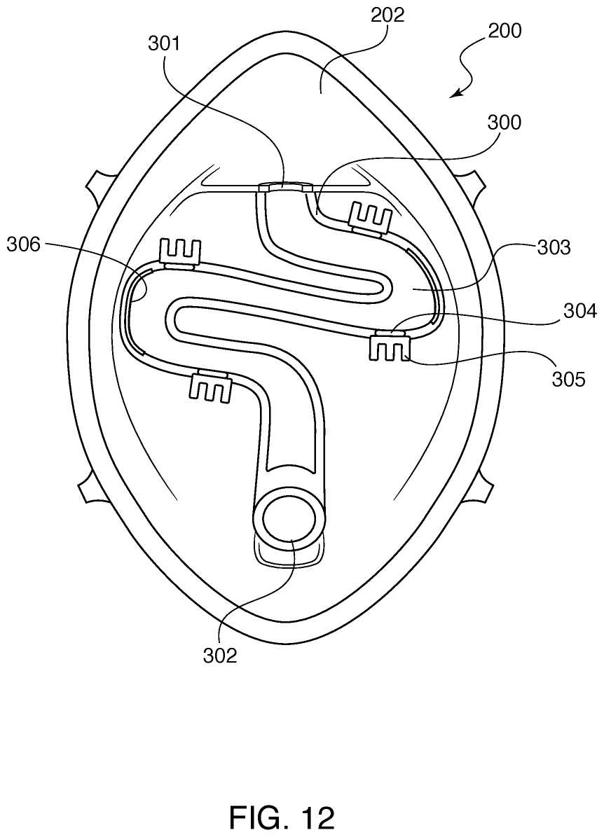

FIG. 10 shows the face mask 200 in the rear view with the face shield 206 removed. A respiration tube 300 is disposed between face shield 206 and outer shell 202. Respiration tube 300 has a serpentine shape with multiple curves, and has an inlet opening 301 that connects to inlet opening 203 on outer shell 202 to allow air flow from outside mask 200 to enter respiration tube 300. Outlet opening 302 is positioned so as to be near the user's mouth and nose, so that air flows from the inlet opening through the respiration tube and out the outlet opening to be breathed in by the user. Exhalations of the user travel into the outlet opening, through the respiration tube and out the inlet opening. Inlet opening 301 and outlet opening 302 are arranged perpendicular to each other, with inlet opening facing upward and outlet opening facing horizontally toward the user's mouth. Respiration tube can be removable from mask 200 or can be integrally molded with outer shell 302. During inhalation and exhalation, the air traveling through the interior 303 of respiration tube 300 is exposed to UV radiation from a series of UV light-emitting diodes (LEDs) 304 arranged inside respiration tube 300, as shown in the cross-sectional views in FIGS. 11 and 12.

LEDs 304 are arranged in different longitudinal areas along respiration tube 300, and on opposite sides of the tube 300, to maximize exposure along the length and width of the tube. Reflectors in the form of mirrors 306 or other types of reflective material are placed at the bends in the respiration tube 300 to further reflect the light from LEDs along the length of the tube. Heat sinks 305 can be placed adjacent each one of LEDs 304 in order to absorb some of the heat generated by LEDs 304 during use. As shown in FIG. 11, respiration tube 300 can have six LEDs 304 arranged along the tube. FIG. 12 shows an embodiment having four LEDs. Although not shown, the LEDs are all connected via wires to wire 200 (shown in FIG. 7) to connect them to battery 223. The wires can be embedded in respiration tube 300 or can be external to respiration tube 300. The user can turn the LEDs on and off either though button 204 or through controller 221. LEDs 304 can also be connected to a temperature-sensitive switch or fuse 308 that disconnects the LED or LEDs in the mask from the power source, either directly or via the controller, in the event that one or more of the LED's exceeds a predetermined temperature.

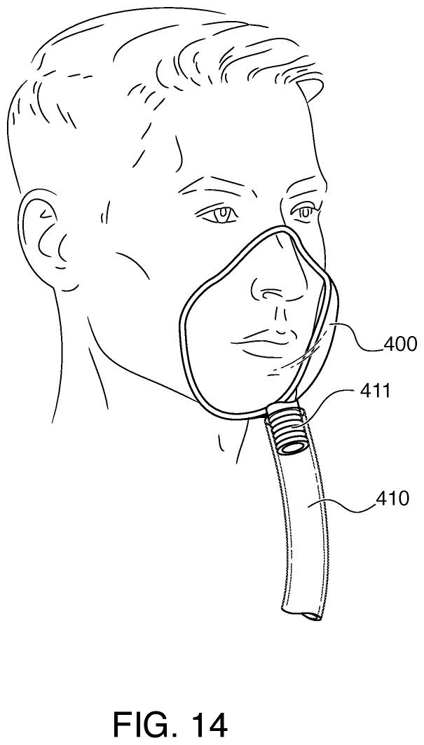

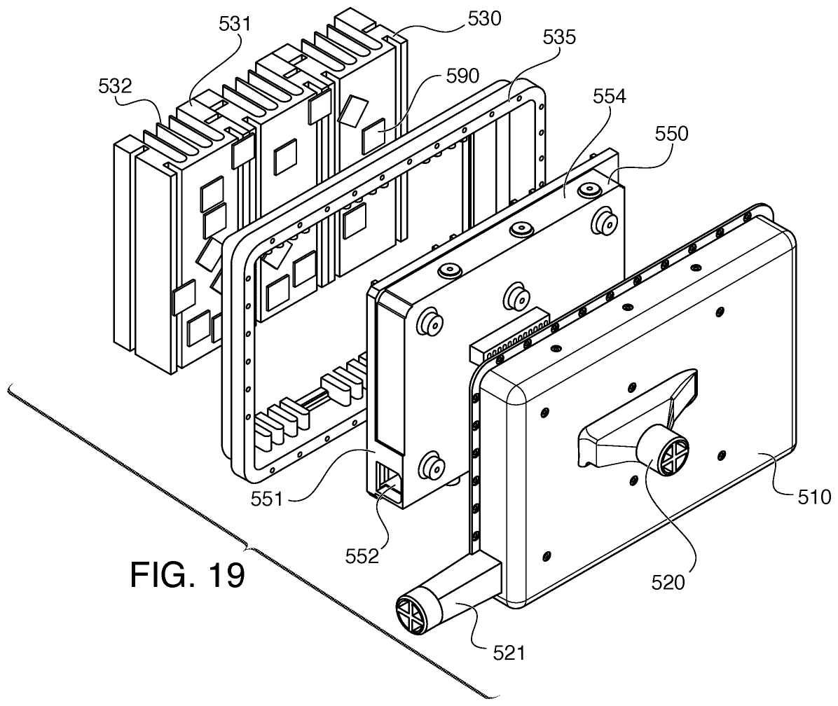

Alternative embodiments of the invention are shown in FIGS. 13-20. Here, instead of having the LEDs mounted in the mask, the LEDs are mounted in a housing and connected by a tube to the mask. For example, FIGS. 13-15 show a first embodiment of this type, in which mask 400 surrounds the user's face and is connected to a tube 410 which leads to a housing 420. A port 411 on mask 400 inserts into tube 410 to connect mask 400 to tube 410.

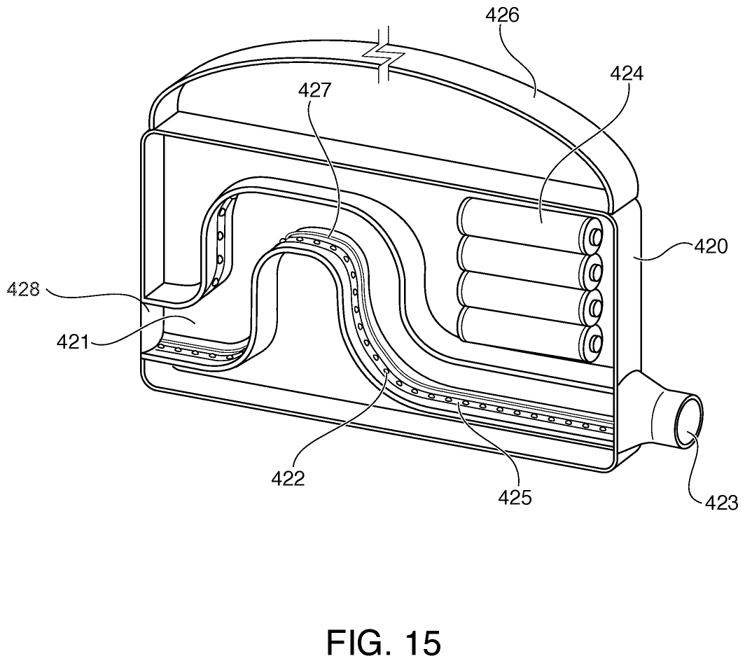

As shown in FIG. 15, inside the housing 420 is a channel 421 equipped with LEDs 422 arranged on a strip 425 that extend throughout its interior. A connection port 423 is located at the end of the channel 421 and connects to tube 410. Opposite connection port 423 is an exit opening 428 that allows ambient air to enter the housing to be inhaled by the user, and allows air exhaled by the user to be released into the environment. The air that is inhaled and exhaled passes through the tube 410 and through channel 421 where it is exposed to LEDs 422 to destroy any microorganisms in the air. The tube 410 could also be divided into two tubes, separated by a valve, for inhalation and exhalation. However, a single tube for both inhalation and exhalation is sufficient in this regard. A battery pack 424 in housing 420 is connected to LEDs 422 by a wire (not shown) to supply power to the LEDs 422. Housing 420 is configured to be lightweight and portable, so that the user can carry it with them throughout their day. A carrying strap 426 can be affixed to the housing. Other attachment and carrying means could also be used. As an alternative or in addition to the LEDs 422, a heating element 427 such as a heat coil can be placed in the channel to heat the air to a temperature that will destroy any microorganisms in the air. The temperature generated is between 60-80° C., which is sufficient to kill the microorganisms, yet low enough to avoid damage to the housing.

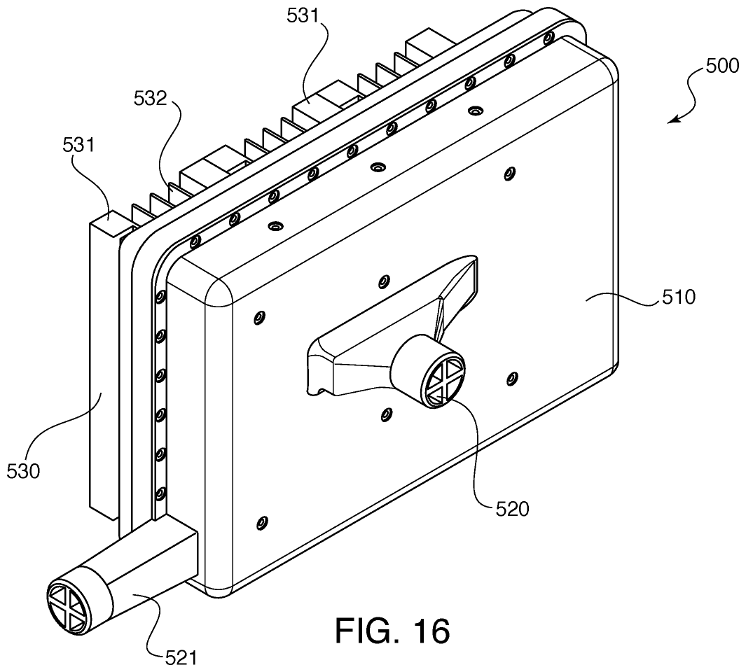

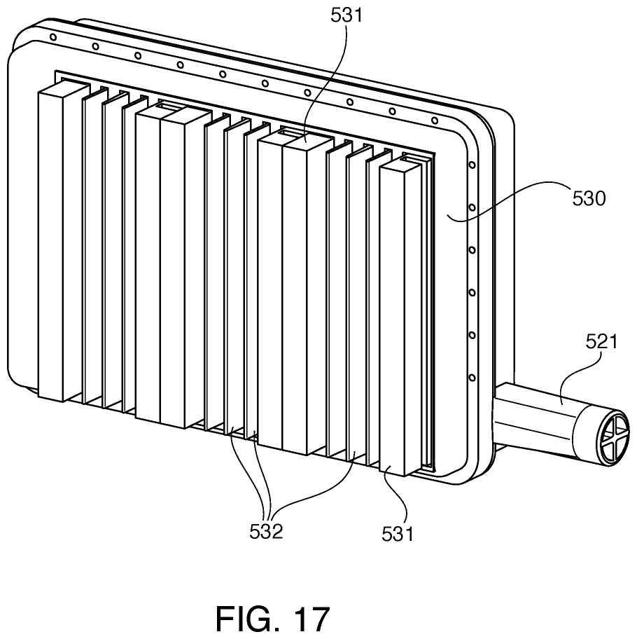

Another embodiment of the housing of the invention is disclosed in FIGS. 16-20. Here, housing 500 is made up of a front panel 510 having an exit port 520, and a connection port 521 for connection to tube 410. A rear panel 530 is surrounded by a frame 535. Rear panel 530 has radiator baffles 532 positioned between spacers 531.

Frame 535 holds rear panel 530 in a removable manner.

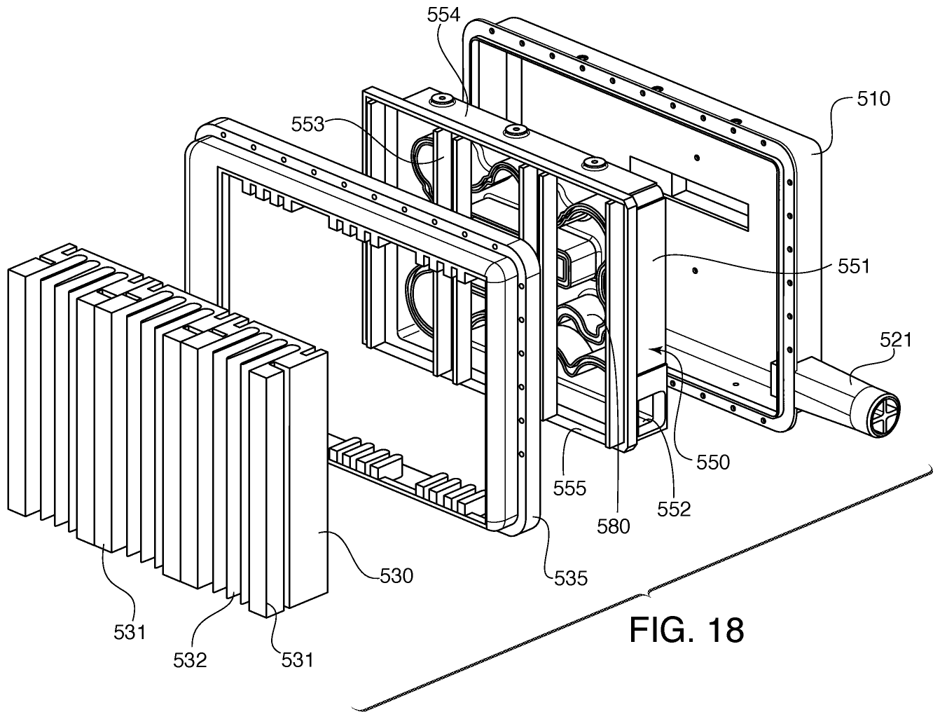

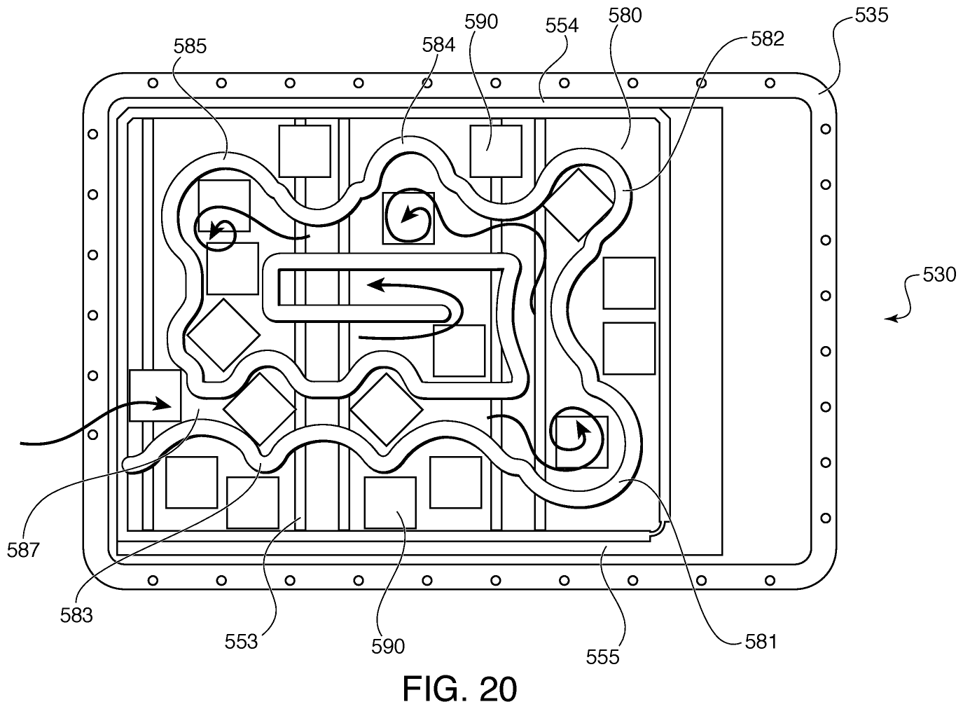

In between front panel 510 and rear panel 530 is a channel plate 550, having a side wall 551 with an opening 552 that opens into connection port 521, and support struts 553 extending between the top and bottom walls 554, 555. A channel wall 580 is positioned on top of struts 553 and forms a helical channel 587 extending from opening 552 to the center of channel plate 550. As can be seen in FIGS. 18 and 20, channel wall 580 has a series of undulations 581, 582, 583, 584, 585 and others. The position of the undulations causes air flowing through the channel to form eddies in the pockets created by the undulations, as can be seen by the helical arrows in FIG. 20. This causes the air to remain in the channel for longer periods of time.

In the assembled position, the center of the channel 587 connects with exit port 520, and the end of channel 587 connects with connection port 521, so that air that is breathed in and out the tube 410 flows through channel 587 and is expelled or inhaled through exit port 520. As the air passes through the channel, it is irradiated by LEDs 590, which are attached to the interior of rear panel 530 so as to be located along the course of the channel 587. LEDs 590 can be powered by any suitable means such as batteries (not shown). The effect of the undulations causes the air to remain the channel for longer periods of time, which increases the exposure of the air to the LEDs, thus more effectively killing any pathogens in the inhaled and exhaled air.

The device of the present invention is a simple and effective way to sanitize the air traveling through a face mask. It is lightweight, portable and inexpensive to manufacture as well as comfortable to wear.

Accordingly, while only a few embodiments of the present invention have been shown and described, it is obvious that many changes and modifications may be made thereunto without departing from the spirit and scope of the invention.

What is claimed is:

1. A sanitizing face mask comprising:
a mask portion having a front surface and a rear surface and being configured to cover a mouth and nose of a wearer, the mask portion having an opening;
a respiration tube having a first end, a second end and an inner space, wherein the first end is connected to the opening and the second end forms an outlet opening;
a housing connected to the outlet opening of the respiration tube, the housing having an interior channel;
at least one ultraviolet (UV) light-source mounted in the housing so as to project UV light into the interior channel; and
a power source connected to the at least one UV light source.

2. The face mask according to claim 1, wherein the power source is at least one battery.

3. The face mask according to claim 1, wherein the UV light source is a UV light-emitting diode (LED).

4. The face mask according to claim 1, wherein there are between four and fifteen LEDs arranged in the housing.

5. The face mask according to claim 1, wherein the LEDs are arranged on a strip.

6. The face mask according to claim 1, wherein the housing has an opening in communication with the interior channel.

7. The face mask according to claim 1, wherein the channel is helical in shape, and connects to the respiration tube at radially outer end of the channel.

8. The face mask according to claim 7, wherein the channel has side walls with a plurality of undulations along the length of the channel, so that a width of the channel varies throughout the length.

9. The face mask according to claim 8, wherein the undulations are configured to form rounded bulges in the channel, said rounded bulges causes air flowing through the channel to form eddies.

10. The face mask according to claim 1, wherein the LEDs are arranged on a back wall of the housing and face the channel.

11. The face mask according to claim 1, wherein the back wall has an exterior surface with a plurality of fins extending therefrom, to allow heat from the LEDs to dissipate during use.

12. The face mask according to claim 1, further comprising a strap connected to the housing.
